(19) United States
(12) Patent Application Publication
(10) Pub. No.: US 2017/0209455 A1
(43) Pub. Date: Jul. 27, 2017

(54) Solid Pharmaceutical Composition Comprising PI3K-Inhibitor

(71) Applicant: NOVARTIS AG, BASEL (CH)

(72) Inventors: Dipen Desai, Whippany, NJ (US); Bruno Galli, Seltisberg (CH); Rakesh Patel, Doylestown, PA (US); Manuel Vincente Sanchez-Felix, West Newbury, MA (US); Frank Hans Seiler, Freiburg (DE); Navnit H. Shah, Clifton, NJ (US); Daya D. Verma, Edison, NJ (US); Toni Widmer, Basel (CH); Stefanie Meyer, Osterried (DE); Raman Iyer, Piscataway, NJ (US); Severine Serreau, Saint-Louis (FR); Simone Grandolini, Basel (CH); Oliver Graner, Basel (CH)

(73) Assignee: Novartis AG, Basel (CH)

(21) Appl. No.: 15/326,790

(22) PCT Filed: Jul. 24, 2015
(86) PCT No.: PCT/IB2015/055622

§ 371 Date: Jan. 17, 2017

Related U.S. Application Data
(60) Provisional application No. 62/173,626, filed on Jun. 10, 2015.
(60) Provisional application No. 62/120,950, filed on Feb. 26, 2015.
(60) Provisional application No. 62/028,917, filed on Jul. 25, 2014.

(51) Int. Cl. A61K 31/5377; A61K 9/48; A61K 9/16

(52) U.S. Cl. CPC A61K 31/5377; A61K 9/1635; A61K 9/1652; A61K 9/1611; A61K 9/1617; A61K 9/1694; A61K 9/4825; A61K 9/4833

(57) ABSTRACT

The application relates to solid pharmaceutical compositions suitable for oral administration, processes for their production and uses of the pharmaceutical compositions.

### FIELD OF THE INVENTION

The present invention relates to solid pharmaceutical dosage forms comprising the compound (4S,5R)-3-(2′-Amino-2-morpholin-4-yl-4′-trifluoromethyl-[4,5]bipyrimidinyl-6-yl)-4-hydroxymethyl-5-methyl-oxazolidin-2-one, or a pharmaceutically acceptable salt thereof, and at least one additional pharmaceutically acceptable carrier. The present invention also relates to the processes for their preparation and to their use as medicaments for the treatment of a proliferative disease, e.g. cancer.

### BACKGROUND OF THE INVENTION

(4S,5R)-3-(2′-Amino-2-morpholin-4-yl-4′-trifluoromethyl-[4,5]bipyrimidinyl-6-yl)-4-hydroxymethyl-5-methyl-oxazolidin-2-one has the structure of formula (I):

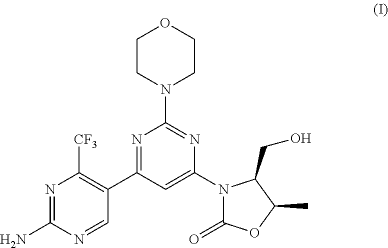 [SMILES: C[C@H]1OC(=O)N(c2cc(-c3cnc(N)nc3C(F)(F)F)nc(N3CCOCC3)n2)[C@H]1CO]

(hereinafter referred to as “compound of formula (I)”). The compound of formula (I) is a Phosphoinositide-3-kinase inhibitor (PI3K inhibitor) and is useful in the suppression of tumors and in other conditions, diseases or disorders dependent on PI3K.

The compound of formula (I), its chemical synthesis, medical use, crystalline and amorphous forms, methods to preparing said forms, and first simple pharmaceutical dosage forms are described in WO 2013/124826, filed Feb. 22, 2013, and are incorporated by reference in its entirety herein.

As the compound of formula (I) is a poorly water soluble compound and difficult to formulate, there is a need to employ special drug delivery technologies to make this compound sufficiently bioavailable.

A first simple solid dispersion comprising compound of formula (I) and hydroxypropylmethylcellulose (HPMC), prepared by a simple solvent removal process, is described in WO 2013/124826, pages 52-53. However, the formulation of WO 2013/124826, which contains a 1:1 w/w ratio of the carrier polymer HPMC (grade 603) to the compound of formula (I), was found by the present inventors to be unstable and subject to changes of the physical state of the compound of formula (I) over time when stored at ambient temperatures. Increasing the weight to weight (w/w) ratio of the HPMC carrier to the compound of formula (I) e.g. up to 4:1, i.e. with only 20% drug load, did not prevent the compound of formula (I) from recrystallizing upon standing.

Therefore, there is still a need for the design of a pharmaceutical dosage form which is able to deliver the compound of formula (I) reliably and at a high rate to achieve high bioavailability. At the same time, the pharmaceutical dosage form should provide for a consistent delivery of the compound of formula (I) during the entire shelf-life of the drug product.

### SUMMARY OF THE INVENTION

The design of a pharmaceutical composition, a pharmaceutical dosage form as well as a commercially viable pharmaceutical manufacturing process for the compound of formula (I) is especially difficult for, inter alia, the following reasons:

Firstly, the compound of formula (I) has a strong tendency to crystallize. In its crystalline form, however, the compound of formula (I) is poorly water-soluble and consequently poorly bioavailable.

Secondly, a high dose of the compound of formula (I) may be needed for therapeutic efficacy in the treatment of some diseases for some of the patients. At the same time, the high dose should fit into a swallowable oral dosage form. Therefore, in such cases, a high drug loading needs to be achieved which in turn limits the amount of excipients which are required to stabilize the compound.

Thirdly, fast delivery of a therapeutic compound out of the pharmaceutical dosage form is preferred for therapy. Therefore, it is desirable that the dosage form is able to release the compound quickly, ideally within a few minutes, preferably within 30 minutes, more preferably within 15 min.

Fourthly, once released out of the dosage form, the compound forms a supersaturated solution. To optimize bioavailability, the compound would ideally stay for a longer time in solution despite its high tendency to crystallize and to precipitate out of aqueous solutions.

Therefore, the compound of formula (I), once released from the pharmaceutical dosage form, should ideally remain in the supersaturated solution for a time period sufficiently long to be absorbed, e.g., into the body of a warm-blooded animal or patient in need of such treatment.

It is therefore difficult to design a pharmaceutical composition or a dosage form for the compound of formula (I) that fulfils these criteria and at the same time is of an acceptable size to be easily swallowable. It is even more difficult to find a pharmaceutical composition for the compound of formula (I) which meets the first two above mentioned requirements and at the same time also ensures fast drug release and long time periods of staying in a supersaturated solution. Further, it is difficult to design a manufacturing process which can reliably produce said pharmaceutical dosage form on a commercial scale and in consistently high quality suitable for human use.

Despite the numerous difficulties associated with the compound of formula (I), the inventors surprisingly found that the first two requirements as stated above can be met when using a stabilizing polymer. The stabilizing polymer is a polymeric material which may be used to embed the compound of formula (I) in its amorphous form in the polymer matrix and is able to keep said compound in its amorphous state over time. Poly(N-vinylpyrrolidone) (PVP) or a derivative thereof were found to be especially suitable stabilizing polymers for the compound of formula (I). Preferably the compound of formula (I) or its pharmaceutically acceptable salt and the stabilizing polymer are in the form of granules.

It was further surprisingly found that when the median particle size of the granules is within 250-1000 μm, the time for the drug release is within 15 minutes. The inventors yet further surprisingly found that an anti-nucleating agent, e.g. an acrylic polymer or a cellulose derived polymer, or combinations thereof, is especially suitable for keeping the compound of formula (I) in supersatured solution. Hydroxypropyl methylcellulose acetate succinate (HPMC-AS) was found to be especially suitable in this aspect.

Taking these surprising findings into account, the inventors herewith provide the present invention as described herein.

In accordance with the present invention, there is provided a pharmaceutical composition comprising the compound (4S,5R)-3-(2′-Amino-2-morpholin-4-yl-4′-trifluoromethyl-[4,5]bipyrimidinyl-6-yl)-4-hydroxymethyl-5-methyl-oxazolidin-2-one or a pharmaceutically acceptable salt thereof and a stabilizing polymer. In accordance with the present invention, there is also provided a pharmaceutical composition comprising the compound (4S,5R)-3-(2′-Amino-2-morpholin-4-yl-4′-trifluoromethyl-[4,5]bipyrimidinyl-6-yl)-4-hydroxymethyl-5-methyl-oxazolidin-2-one and a stabilizing polymer. In the embodiments of the present invention said stabilizing polymer is preferably poly(N-vinylpyrrolidone) (PVP), or a derivative thereof, more preferably, said stabilizing polymer is a copolymer of N-vinylpyrrolidone and vinylacetate.

More specifically there is provided a pharmaceutical composition as described above, wherein the drug substance, i.e the compound of formula (I) or its pharmaceutically acceptable salt, and the stabilizing polymer are present in the form of granules. There is provided a pharmaceutical composition as described above, wherein the drug substance, i.e the compound of formula (I) or its pharmaceutically acceptable salt, and the stabilizing polymer are present together as a mixture in the form of granules.

The granules described herein may be present in the pharmaceutical compositions provided herein in about 15-70%, e.g., preferably about 15-50%, e.g, preferably about 5-15% by weight, based on the total weight of the granules and all extragranular ingredients together.

Even more specifically there is provided a pharmaceutical composition as described above, wherein said granules comprise about 5-50%, preferably about 10-40%, more preferably about 30-35% by weight of the compound of formula (I) based on the total weight of said granules.

Even more specifically there is provided a pharmaceutical composition as described above, wherein the granules have a median particle size within 250 to 1000 μm, preferably within 300 to 750 μm, more preferably within 300 to 500 μm.

In one embodiment, there is provided a pharmaceutical composition as described above, further comprising an anti-nucleating agent, preferably said anti-nucleating agent is an acrylic polymer or a cellulose-derived polymer, or combinations thereof, more preferably said anti-nucleating agent is selected from the group consisting of methacrylic acid-methyl methacrylate copolymer 1:1 (Eudragit L100), hydroxypropyl methylcellulose (HPMC) and hydroxypropyl methylcellulose acetate succinate (HPMC-AS), and combinations thereof. Even more preferably said anti-nucleating agent is hydroxypropyl methylcellulose acetate succinate (HPMC-AS). It is understood that the pharmaceutical composition of the invention may or may not comprise an anti-nucleating agent.

In accordance with the present invention, there is further provided a process for preparation of the pharmaceutical composition as described above comprising a melt granulation, preferably a melt-extrusion step.

In accordance with the present invention, there is further provided a pharmaceutical composition obtainable by the process as described above.

In accordance with the present invention, there is further provided a pharmaceutical composition as described above for use in the treatment of cancer and/or the treatment or prevention of other conditions, diseases or disorders dependent on PI3K.

The pharmaceutical compositions of the present invention provide for a drug product which is physically stable during storage and during drug dissolution. The pharmaceutical compositions of the present invention also provide a reliably high bioavailability of the therapeutic agent which is (4S,5R)-3-(2′-Amino-2-morpholin-4-yl-4′-trifluoromethyl-[4,5]bipyrimidinyl-6-yl)-4-hydroxymethyl-5-methyl-oxazolidin-2-one.

### BRIEF DESCRIPTION OF THE FIGURE

FIG. 1 shows the in vitro drug dissolution profiles of capsules comprising the compound of formula (I) embedded in copovidone (Kollidon VA 64) as melt extrudates (internal phase) and different external stabilizers in the external phase. The FIGURE demonstrates that 10% HPMC Acetate Succinate is especially effective in keeping the compound of formula (I) in solution.

### DETAILED DESCRIPTION OF THE INVENTION

Herein after, the present invention is described in further detail and is exemplified. It will be understood that the features described herein may be combined together.

In one aspect of the present invention, there is provided a pharmaceutical composition comprising the compound (4S,5R)-3-(2′-Amino-2-morpholin-4-yl-4′-trifluoromethyl-[4,5]bipyrimidinyl-6-yl)-4-hydroxymethyl-5-methyl-oxazolidin-2-one or a pharmaceutically acceptable salt and a stabilizing polymer.

In the aspects of the pharmaceutical composition of the present invention, the compound (4S,5R)-3-(2′-Amino-2-morpholin-4-yl-4′-trifluoromethyl-[4,5]bipyrimidinyl-6-yl)-4-hydroxymethyl-5-methyl-oxazolidin-2-one, herein also referred to as compound of formula (I), is present in its free form or in the form of any pharmaceutically acceptable salt, complex, co-crystal, hydrate or solvate thereof, preferably in its free form.

The compound of formula (I) is present in its amorphous state. The phrase “present in its amorphous state” has herein the meaning of “present to a substantial amount in the amorphous state.” Preferably the amorphous state is characterized by the absence of said compound in any of its crystalline states. The absence of crystalline compound of formula (I) can be determined by x-ray powder diffraction (XRPD) and/or differential scanning calorimetry (DSC). The amorphous state is further characterized by having in an XRPD or DSC analysis not more than 50%, preferably not more than 25%, more preferably not more than 10%, even more preferably not more than 5%, even more preferably not more than 2%, even more preferably not more than 1%, even more preferably not more than 0.5% of compound of formula (I) in any of its crystalline states based on the total amount of the compound of formula (I) in a dose unit of the pharmaceutical composition. Most preferably, the compound of formula (I) is entirely present (to 100%) in its amorphous state, i.e. not present in any of its crystalline states. The compound of formula (I) in amorphous form may be prepared by melt extrusion with a suitable stabilizing polymer, e.g. copovidone as described herein or by spray drying a mixture of the compound of formula (I) in a suitable solvent or solvent mixture such as methanol, ethanol, isopropanol, n-butanol, isobutanol, methylene chloride, chloroform, acetone or combinations thereof.

The stabilizing polymer stabilizes very efficiently the amorphous state of the drug substance so that a granule drug load of about 30 to 35% or even higher is achievable. This in turn makes the resulting drug product more easily swallowable by patients.

The stabilizing polymer may be poly(N-vinylpyrrolidone) (PVP) (also referred to as povidone), or a derivative thereof such as cross-linked PVP (also referred to as crospovidone), copolymers of N-vinylpyrrolidone and vinyl acetate (also referred to as copovidone), or a physical mixture of polyvinyl acetate and povidone.

Examples of povidones are Kollidon products supplied by BASF which are available with different K-values, e.g. 12, 17, 25, 30, 90. Examples of crospovidones are Kollidon products supplied by BASF which are available in different grades, e.g. CL (standard), CL-F (fine), CL-SF (super fine), CL-M (micronized). An example of a copovidone is Kollidon VA 64 supplied by BASF which contains the N-vinylpyrrolidone and vinylacetate in a mass ratio of 6:4. The Kollidon VA 64 is available in different grades, e.g. VA 64 (standard), VA 64 Fine (fine). An example of a physical mixture of polyvinyl acetate (PVAc) and povidone is of Kollidon SR supplied by BASF which is a mixture of PVAc and povidone 30 in the ratio of 8:2 with small amounts of sodium lauryl sulphate and silica. All these povidones, crospovidones, copovidones and PVA-povidone mixtures are described in detail in Volker Bühler, “Kollidon—Polyvinylpyrrolidone excipients for the pharmaceutical industry”, BASF, 9^{th} revised edition, March 2008, which is incorporated by reference herein in its entirety.

In one embodiment the stabilizing polymer is a povidone such as Kollidon K12 or K30, or an equivalent thereof.

In another embodiment the stabilizing polymer is a copovidone such as Kollidon VA 64.

In yet another embodiment, said stabilizing polymer is a combination of different povidone types, or a combination of a copovidone with a povidone.

In a preferred embodiment, the stabilizing polymer is a copovidone, more preferably the copovidone is a copovidone such as Kollidone VA 64 or an equivalent thereof.

The compound of formula (I), or a pharmaceutically acceptable salt thereof, the stabilizing polymer, and optionally any further ingredients or excipients may be present together in the form of granules and form an internal phase or intragranular phase of the drug product. The ingredients of the granules are also referred to as internal or intragranular ingredients. In other words, the compound of formula (I), or a pharmaceutically acceptable salt thereof, and the polymer are present in the intragranular phase or are present intragranularly. In one embodiment, the compound of formula (I) or a pharmaceutically acceptable salt thereof, the stabilizing polymer, and a glidant (e.g., colloidal silicon dioxide) are present in the intragranular phase of the drug product.

Said granules alone may already form the drug product, e.g. in the form of small pellets which are filled into capsules or in the form of lentils directly made from melt-extrusion.

Alternatively, the granules are combined with further ingredients which then form the drug product.

Said further ingredients which do not form part of the granules are referred to as “extragranular” ingredients and constitutes the external phase or extragranular phase.

The granules may be prepared by dry granulation or wet granulation. Alternatively, the granules may be obtained by melt granulation, or melt extrusion. As further alternatives, the granules may be obtained by simple solvent removal processes or by spray drying. Preferably, the granules are obtained by hot melt extrusion and are subsequently milled to a desired particle size.

The granules, if present, may comprise of about 5-50%, e.g., about 10-40%, e.g., about 30-35%, preferably about 10-40%, more preferably about 30-35% by weight of the compound of formula (I) based on the total weight of said granules. It is a surprising finding of the present invention that such a high amount of the therapeutically active compound is stabilized in its amorphous form by the use of the polymers as described herein and provide a pharmaceutical composition wherein the compound has a high kinetic solubility.

The granules are of a mean or median particle size of 250-1000 μm, preferably 300-750 μm, more preferably of 350-500 μm as determined by sieve analysis. In one preferred embodiment, the granules are of a median particle size of 250-1000 μm, In one preferred embodiment, the granules are of a median particle size of 300-750 μm, more preferably of 350-500 μm as determined by sieve analysis.

In another embodiment the granules are characterized in that at least 50% by weight of the particles are larger than 250 μm but at least 90% by weight are smaller than 1000 μm. In other words, at least 50% by weight of the particles do not pass a sieve with a mesh size corresponding to 250 μm and at least 90% by weight pass a sieve with a mesh size corresponding to 1000 μm.

Granules with a mean or median particle size or particles size distribution as described herein have the advantage that they do not induce an undesired gelation effect when the final drug product is exposed to aqueous media for dissolution. The formation of a gel would retard the drug release in an undesired way.

The pharmaceutical composition of the present invention may further contain an anti-nucleating agent. The anti-nucleating agent may be an acrylic polymer or a cellulose derived polymer or combinations thereof. The anti-nucleating agent may be selected from the group consisting of methacrylic acid-methyl methacrylate copolymer 1:1 (Eudragit L100), hydroxypropyl methylcellulose (HPMC) and hydroxypropyl methylcellulose acetate succinate HPMC-AS, or combinations thereof. Even more preferably said anti-nucleating agent is hydroxypropyl methylcellulose acetate succinate (HPMC-AS). It is understood that the pharmaceutical composition of the invention may or may not comprise an anti-nucleating agent.

Acrylic polymers are polymers or copolymers which are composed e.g. of acrylic acid, methacrylic acid, methyl acrylate, or methyl methacrylate monomers or combinations of those monomers. Further, all derivatives of those polymers are also understood to be included in the group of acrylic polymers. Examples of this group of polymers are those which are commercialized under the brand name Eudragit or Eudragid by Evonik Industries, e.g. Eudragit L 100, Eudragit L 12,5, Eudragit S 100, Eudragit S 12,5, Eudragit L 100-55, Eudragit RL 100, Eudragit RL PO, Eudragit RL 12,5, Eudragit RS 100, Eudragit RS PO, Eudragit RS 12,5, Eudragit E 100, Eudragit E PO, Eudragit E 12,5.

Cellulose derivative polymers herein are e.g. methylcellulose, ethylcellulose, hydroxypropylcellulose, hydroxypropylmethylcellulose, more preferably a hydroxypropylmethylcellulose (HPMC, also referred to as hypromellose), even more preferably HPMC acetate succinate (HPMC-AS) in any of its available grades, e.g. micronized grades such as AS-LF, AS-MF, AS-HF or granular grades such as AS-LG, AS-MG, AS-HG as e.g. commercialized by ShinEtsu Chemical Co., even more preferred is the grade HPMC-AS-LF (also referred to as HPMC-AS, LF), which typically has about 8% acetyl content (5-9%), about 15% succinoyl content (14-18%), a mean particle size of about 5 μm (average: not more than 10 μm, 90% cumulation: not more than 20 μm) and a viscosity of about 3 mm²/s (2.4-3.6 mm²/S), methoxy content of 20-24%, hydroxypropoxy content of 5-9%.

The term “anti-nucleating agent” is used herein in its established meaning in the field of pharmaceutics, i.e. is a compound which is able to reduce the rate of re-crystallization or prevent the recrystallization of another compound, here the drug substance, i.e. the compound of formula (I). In its role the anti-nucleating agent stabilizes the supersaturated solution which is obtained when the drug product gets into contact with aqueous media and prevents that the compound quickly precipitates again.

The pharmaceutical composition of the present invention may further comprise conventional excipients. Examples of such conventional excipients include, but are not limited to, fillers, disintegrants, lubricants, and glidants.

The pharmaceutical composition of the present invention may further comprise a filler such as lactose, sucrose, glucose, mannitol, sorbitol, calcium phosphate, calcium carbonate, cellulose or any combination thereof, preferably the filler is a cellulose, more preferably the filler is microcrystalline cellulose (also referred to as MCC or cellulose MK GR). It was surprisingly found that the microcrystalline cellulose acted as spacer and increased the porosity of the final drug product. In this role the microcrystalline cellulose contributed to the prevention of the undesired formation of a gel which in turn would reduce drug dissolution rate.

In one embodiment, the pharmaceutical composition of the present invention comprises microcrystalline cellulose having a nominal particle size of about 100 μm (e.g., Vivapur 102, Avicel PH-102). In a further embodiment, the pharmaceutical composition of the present invention comprises microcrystalline cellulose having a nominal particle size of about 50-70 μm (e.g., Vivapur 101, Avicel PH-101). In a further embodiment, the pharmaceutical composition of the present invention comprises microcrystalline cellulose having a nominal particle size of about 50 μm (e.g., Vivapur 101, Avicel PH-101) and microcrystalline cellulose having a nominal particle size of about 100 μm (e.g., Vivapur 102, Avicel PH-102).

The pharmaceutical composition of the present invention may further comprise a disintegrant such as starch, cellulose, cross-linked poly(N-vinylpyrrolidone), sodium starch glycolate, sodium carboxymethyl cellulose (e.g., croscarmellose sodium) or combinations thereof, preferably cross-linked poly(N-vinylpyrrolidone) (also referred to as PVP-XL or crospovidone) or sodium carboxymethyl cellulose (e.g., croscarmellose sodium).

The pharmaceutical composition of the present invention may further comprise a lubricant, a glidant or a combination thereof. Examples of lubricants and glidants include, colloidal silicon dioxide, magnesium trisilicate, starches, talc, tribasic calcium phosphate, magnesium stearate, aluminum stearate, calcium stearate, sodium stearyl fumarate, magnesium carbonate, magnesium oxide, polyethylene glycol, powdered cellulose and microcrystalline cellulose. A preferred lubricant is magnesium stearate or sodium stearyl fumarate. A preferred glidant is colloidal silicon dioxide.

The additional excipients, including but not limited to the anti-nucleating agent, the filler, the disintegrant, the lubricant, the glidant and combinations thereof, may be present extragranularly, i.e they do not form part of the granular matrix which comprises the compound of formula (I) and the stabilizing polymer. It is understood that a specific additional excipient may be present both intragranularly and extragranularly. Preferably, the anti-nucleating agent, if present, is present extragranularly only.

More specifically, the pharmaceutical composition of any of the embodiments as described herein is characterized in that the hydroxypropyl methylcellulose acetate succinate (HPMC-AS), where present, is present in about 5-15%, preferably in about 7-10% by weight of the total weight of the pharmaceutical composition (i.e the weight of the granules and the weight of the extragranular ingredients).

More specifically, the pharmaceutical composition of any of the embodiments as described herein is characterized in that the filler, preferably microcrystalline cellulose, where present, is present in about 30-85%, e.g. about 30-80% e.g., about 50-85%, e.g., about 20-75%, e.g., about 40-70%, preferably in about 40-70% by weight of the total weight of the pharmaceutical composition (i.e the weight of the granules and the weight of the extragranular ingredients).

More specifically, the pharmaceutical composition of any of the embodiments as described herein is characterized in that the disintegrant, where present, is present in about 5-20%, preferably in about 5-18%, more preferably in about 10-15% by weight of the total weight of the pharmaceutical composition (i.e the weight of the granules and the weight of the extragranular ingredients).

More specifically, the pharmaceutical composition of any embodiment as described herein is characterized in that the lubricant, where present, is present in about 0.1-5%, preferably about 0.1-2% by weight of the total weight of the pharmaceutical composition (i.e the weight of the granules and the weight of the extragranular ingredients).

More specifically, the pharmaceutical composition of any embodiment as described herein is characterized in that the glidant, where present, is present in about 0.01-7%, preferably about 1-6% by weight of the total weight of the pharmaceutical composition (i.e the weight of the granules and the weight of the extragranular ingredients).

The pharmaceutical composition of any of the embodiments as described herein is characterized in that the composition is in the form of a solid pharmaceutical dosage form, including without limitation, capsules, tablets, caplets, granules, and sachets, preferably a capsule or tablet. In some aspects, granules and tablets may be coated with a suitable polymer or a conventional coating material to achieve, for example, greater stability in the gastrointestinal tract, or to achieve the desired rate of release. Suitable film coatings are known and commercially available or can be made according to known methods. The film coating may be applied by conventional techniques in a suitable coating pan or fluidized bed apparatus using water and/or conventional organic solvents (e.g, methyl alcohol, ethyl alcohol, isopropyl alcohol), ketones (acetone), etc.

Moreover, capsules containing the pharmaceutical composition of the present invention may be further coated. Tablets may be scored to facilitate division of dosing. Alternatively, the dosage forms of the present invention may be unit dosage forms wherein one unit dosage form is intended to deliver one therapeutic dose per administration or wherein multiple unit dosage forms are intended to deliver the total therapeutic dose per administration.

In a preferred embodiment the pharmaceutical composition comprises, substantially consists of, or consists of
(a) 5-15%, preferably 9.5±2% by weight of granules which in turn comprise, substantially consist of, or consist of
   10-40%, preferably 35±5% by weight of the compound of formula (I);
   60-90%, preferably 64.5±10% by weight of copovidone; and
   0.1-2%, preferably 0.5±0.3% by weight colloidal silicon dioxide;
   based on the total weight of the granules;
(b) 50-85%, preferably 66.5±10% by weight of microcrystalline cellulose;
(c) 5-15%, preferably 10±2% by weight of crospovidone;
(d) 5-15%, preferably 10±2% by weight of HPMC acetate succinate;
(e) 1-5%, preferably 3±1% by weight of colloidal silicon dioxide; and
(f) 0.5-2%, preferably 1±0.5% by weight of magnesium stearate;
based on the total weight of granules and all extragranular ingredients together.

The present invention also provides a pharmaceutical composition which comprises, substantially consists of, or consists of
(a) 9.5±2% by weight of granules which in turn comprise, substantially consist of, or consist of
   35±5% by weight of the compound of formula (I);
   64.5±10% by weight of copovidone; and
   0.5±0.3% by weight colloidal silicon dioxide;
   based on the total weight of the granules;
(b) 66.5±10% by weight of microcrystalline cellulose;
(c) 10±2% by weight of crospovidone;
(d) 10±2% by weight of HPMC acetate succinate;
(e) 3±1% by weight of colloidal silicon dioxide; and
(f) 1±0.5% by weight of magnesium stearate;
based on the total weight of granules and all extragranular ingredients together.

In another preferred embodiment the pharmaceutical composition comprises, substantially consists of or consists of
(a) 15-70%, preferably 35.7±5% by weight of granules which in turn comprise, substantially consist of, or consist of
   10-40%, preferably 35±5% by weight of the compound of formula (I);
   60-90%, preferably 64.5±10% by weight of copovidone; and
   0.1-2%, preferably 0.5±0.3% by weight colloidal silicon dioxide;
   based on the total weight of the granules;
(b) 20-75%, preferably 40.3±10% by weight of microcrystalline cellulose;
(c) 5-15%, preferably 10±2% by weight of crospovidone;
(d) 5-15%, preferably 10±2% by weight of HPMC acetate succinate;
(e) 1-5%, preferably 3±1% by weight of colloidal silicon dioxide; and
(f) 0.5-2%, preferably 1±0.5% by weight of magnesium stearate;
based on the total weight of granules and all extragranular ingredients together.

In another preferred embodiment the pharmaceutical composition comprises, substantially consists of or consists of
(a) 15-70%, preferably 35.7±5% by weight of granules which in turn comprise, substantially consist of, or consist of
   10-40%, preferably 35±5% by weight of the compound of formula (I);
   60-90%, preferably 64.5±10% by weight of copovidone; and
   0.1-2%, preferably 0.5±0.3% by weight colloidal silicon dioxide;
   based on the total weight of the granules;
(b) 20-75%, preferably 40.3±10% by weight of microcrystalline cellulose;
(c) 5-15%, preferably 10±2% by weight of crospovidone;
(d) 5-15%, preferably 10±2% by weight of HPMC acetate succinate;
(e) 1-5%, preferably 3±1% by weight of colloidal silicon dioxide; and
(f) 0.5-2%, preferably 1±0.5% by weight of magnesium stearate;
based on the total weight of granules and all extragranular ingredients together.

In a preferred embodiment the pharmaceutical composition comprises, substantially consists of, or consists of
(a) 35.7±5% by weight of granules which in turn comprise, substantially consist of, or consist of
   35±5% by weight of the compound of formula (I);
   64.5±10% by weight of copovidone; and
   0.5±0.3% by weight colloidal silicon dioxide;
   based on the total weight of the granules;
(b) 40.3±10% by weight of microcrystalline cellulose;
(c) 10±2% by weight of crospovidone;
(d) 10±2% by weight of HPMC acetate succinate;
(e) 3±1% by weight of colloidal silicon dioxide; and
(f) 1±0.5% by weight of magnesium stearate;
based on the total weight of granules and all extragranular ingredients together.

In another preferred embodiment the pharmaceutical composition comprises, substantially consists of or consists of
(a) 15-70%, preferably 22.3±5% by weight of granules which in turn comprise, substantially consist of, or consist of
   10-40%, preferably 35±5% by weight of the compound of formula (I);
   60-90%, preferably 64.5±10% by weight of copovidone; and
   0.1-2%, preferably 0.5±0.3% by weight colloidal silicon dioxide;
   based on the total weight of the granules;
(b) 20-75%, preferably 57.2±10% by weight of microcrystalline cellulose;
(c) 5-15%, preferably 10.2±2% by weight of sodium carboxymethyl cellulose;
(d) 5-15%, preferably 7.8±2% by weight of HPMC acetate succinate;
(e) 1-5%, preferably 1.9±1% by weight of colloidal silicon dioxide; and
(f) 0.5-2%, preferably 0.6±0.5% by weight of magnesium stearate;
based on the total weight of granules and all extragranular ingredients together.

In a preferred embodiment the pharmaceutical composition comprises, substantially consists of, or consists of
(a) 22.3±5% by weight of granules which in turn comprise, substantially consist of, or consist of
   35±5% by weight of the compound of formula (I);
   64.5±10% by weight of copovidone; and
   0.5±0.3% by weight colloidal silicon dioxide;
   based on the total weight of the granules;
(b) 57.2±10% by weight of microcrystalline cellulose;
(c) 10.2±2% by weight of sodium carboxymethyl cellulose;
(d) 7.8±2% by weight of HPMC acetate succinate;
(e) 1.9±1% by weight of colloidal silicon dioxide; and
(f) 0.6±0.5% by weight of magnesium stearate;
based on the total weight of granules and all extragranular ingredients together.

In another preferred embodiment the pharmaceutical composition comprises, substantially consists of or consists of
(a) 15-70%, preferably 26±5% by weight of granules which in turn comprise, substantially consist of, or consist of
   10-40%, preferably 35±5% by weight of the compound of formula (I);
   60-90%, preferably 64.5±10% by weight of copovidone; and
   0.1-2%, preferably 0.5±0.3% by weight colloidal silicon dioxide;
   based on the total weight of the granules;
(b) 20-75%, preferably 45.8±10% by weight of microcrystalline cellulose;
(c) 5-18%, preferably 15±2% by weight of sodium carboxymethyl cellulose;
(d) 5-15%, preferably 10±2% by weight of HPMC acetate succinate;
(e) 1-5%, preferably 2.4±1% by weight of colloidal silicon dioxide; and
(f) 0.5-2%, preferably 0.8±0.5% by weight of magnesium stearate;
based on the total weight of granules and all extragranular ingredients together.

In a preferred embodiment the pharmaceutical composition comprises, substantially consists of, or consists of
(a) 26±5% by weight of granules which in turn comprise, substantially consist of, or consist of
   35±5% by weight of the compound of formula (I);
   64.5±10% by weight of copovidone; and
   0.5±0.3% by weight colloidal silicon dioxide;
   based on the total weight of the granules;
(b) 45.8±10% by weight of microcrystalline cellulose;
(c) 15±2% by weight of sodium carboxymethyl cellulose;
(d) 10±2% by weight of HPMC acetate succinate;
(e) 2.4±1% by weight of colloidal silicon dioxide; and
(f) 0.8±0.5% by weight of magnesium stearate;
based on the total weight of granules and all extragranular ingredients together.

In a preferred embodiment the pharmaceutical composition comprises, substantially consists of, or consists of
(a) 15-70%, preferably 34.7±2% by weight of granules which in turn comprise, substantially consist of, or consist of
   10-40%, preferably 30±5% by weight of the compound of formula (I);
   60-90%, preferably 69.5±10% by weight of copovidone; and
   0.1-2%, preferably 0.5±0.3% by weight colloidal silicon dioxide;
   based on the total weight of the granules;
(b) 20-75%, preferably 52.5±10% by weight of microcrystalline cellulose;
(c) 5-15%, preferably 10±2% by weight of crospovidone;
(d) 1-5%, preferably 1.5±0.5% by weight of colloidal silicon dioxide; and
(e) 0.5-2%, preferably 1.25±0.5% by weight of sodium stearyl fumarate;
based on the total weight of granules and all extragranular ingredients together.

The present invention also provides a pharmaceutical composition which comprises, substantially consists of, or consists of
(a) 34.7±2% by weight of granules which in turn comprise, substantially consist of, or consist of
   30±5% by weight of the compound of formula (I);
   69.5±10% by weight of copovidone; and
   0.5±0.3% by weight colloidal silicon dioxide;
   based on the total weight of the granules;
(b) 52.5±10% by weight of microcrystalline cellulose;
(c) 10±2% by weight of crospovidone;
(d) 1.5±0.5% by weight of colloidal silicon dioxide; and
(e) 1.25±0.5% by weight of sodium stearyl fumarate;
based on the total weight of granules and all extragranular ingredients together.

For each embodiment of the present invention, it is understood that the total amount of all components in the granules must add up to 100% by weight based on the total weight of granules. For each embodiment of the present invention, it is further understood that the total amount of the granule and all other components (excluding the granule) in the pharmaceutical composition must add up to 100% by weight based on the total weight of granules and all extragranular ingredients together.

In another aspect of the present invention there is provided a process for making the pharmaceutical composition as described above comprising a melt granulation step.

In a preferred embodiment the melt granulation is performed as hot melt extrusion with subsequent milling of the melt extrudates to a desired particle size.

In one embodiment, the process of the present invention is characterized by the following process steps:
(1) melt-granulating or melt-extruding, preferably melt-extruding the compound (4S,5R)-3-(2′-Amino-2-morpholin-4-yl-4′-trifluoromethyl-[4,5]bipyrimidinyl-6-yl)-4-hydroxymethyl-5-methyl-oxazolidin-2-one or any pharmaceutically acceptable salt thereof together with the polymer poly(N-vinylpyrrolidone) (PVP) or any derivative thereof, preferably, said polymer is a copolymer of N-vinylpyrrolidone and vinylacetate, and optionally colloidal silica;
(2) milling and/or screening the melt granulates obtained by step (1) to obtain melt granulates with a median particle size median particle size within 250 to 1000 μm, preferably within 300 to 750 μm, more preferably within 300 to 500 μm;
(3) blending the melt granulates obtained by step (2) with an anti-nucleating agent, preferably said anti-nucleating agent is an acrylic polymer or a cellulose derived polymer or combinations thereof, more preferably said anti-nucleating agent is selected from the group consisting of Eudragit L100, HPMC and HPMC-AS, even more preferably said anti-nucleating agent is hydroxypropyl methylcellulose acetate succinate (HPMC-AS), and optionally microcrystalline cellulose, and optionally a disintegrant, preferably, cross-linked poly(N-vinylpyrrolidone) or sodium carboxymethyl cellulose, and optionally a glidant, and optionally a lubricant;
(4) encapsulating or tabletting the blend obtained by step (3), preferably encapsulating said blend into hard gelatine capsules.

In a further embodiment, the process of the present invention is characterized by the following process steps:
(1) melt-granulating or melt-extruding, preferably melt-extruding the compound (4S,5R)-3-(2′-Amino-2-morpholin-4-yl-4′-trifluoromethyl-[4,5]bipyrimidinyl-6-yl)-4-hydroxymethyl-5-methyl-oxazolidin-2-one or any pharmaceutically acceptable salt thereof together with the polymer poly(N-vinylpyrrolidone) (PVP) or any derivative thereof, preferably, said polymer is a copolymer of N-vinylpyrrolidone and vinylacetate, and optionally colloidal silica;
(2) milling and/or screening the melt granulates obtained by step (1) to obtain melt granulates with a median particle size median particle size within 250 to 1000 μm, preferably within 300 to 750 μm;
(3) optionally blending the melt granulates obtained by step (2) with one or more excipients, including but not limited to microcrystalline cellulose, a disintegrant, preferably, crospovidone; a glidant, a lubricant, or a combination thereof; and
(4) encapsulating or tabletting the blend obtained by step (3), preferably tableting said blend into tablets.

In alternative embodiments, the compound of formula (I) may be co-processed in the process step (1) with the polymer and optionally the colloidal silicon dioxide by spray drying, by spray congealing, or by the use of suitable solvents in connection with a solvent removal process, e.g. freeze drying.

In yet another aspect of the present invention there is provided pharmaceutical composition obtainable by the process as described above.

The present invention also provides a pharmaceutical composition as described above for use in the treatment of cancer, or for use in the treatment or suppression of tumors, or for use in the treatment or prevention of other conditions, diseases or disorders dependent on PI3K.

The present invention also provides a method for treating cancer in a subject in need of such treatment, which method comprises administering to said subject an effective amount of a pharmaceutical composition as defined above.

The present invention also provides a method for treating or suppressing tumors in a subject in need of such treatment, which method comprises administering to said subject an effective amount of a pharmaceutical composition as defined above.

The present invention also provides a method for treating or preventing other conditions, diseases or disorders dependent on PI3K, in a subject in need of such treatment, which method comprises administering to said subject an effective amount of a pharmaceutical composition as defined above. Various enumerated embodiments of the invention are described herein. It will be recognized that features specified in each embodiment may be combined with other specified features to provide further embodiments of the present invention

### Embodiment 1

A pharmaceutical composition comprising the compound of formula (I), also known as (4S,5R)-3-(2′-Amino-2-morpholin-4-yl-4′-trifluoromethyl-[4,5]bipyrimidinyl-6-yl)-4-hydroxymethyl-5-methyl-oxazolidin-2-one, or a pharmaceutically acceptable salt thereof, and a stabilizing polymer.

### Embodiment 2

The pharmaceutical composition according to Embodiment 1, wherein the stabilizing polymer is poly(N-vinylpyrrolidone) (PVP) or a derivative thereof, preferably, said polymer is a copolymer of N-vinylpyrrolidone and vinylacetate.

### Embodiment 3

The pharmaceutical composition according to Embodiments 1 or 2, wherein the compound of formula (I) or a pharmaceutically acceptable salt thereof, and the stabilizing polymer are present in the form of granules.

### Embodiment 4

The pharmaceutical composition according to Embodiment 3, wherein the granules comprise about 5-50%, preferably about 10-40%, more preferably about 30
35 by weight of the compound of formula (I) based on the total weight of said granules.

### Embodiment 5

The pharmaceutical composition according to any one of Embodiments 3 or 4, wherein the granules have a median particle size ranging from 250 to 1000 μm, preferably ranging from 300 to 750 μm, more preferably ranging from 300 to 500 μm.

### Embodiment 6

The pharmaceutical composition according to Embodiment 5, wherein the granules further comprise a glidant.

### Embodiment 7

The pharmaceutical composition according to any one of Embodiments 1 to 6, further comprising an anti-nucleating agent.

### Embodiment 8

The pharmaceutical composition according to Embodiment 7, wherein the anti-nucleating agent is an acrylic polymer or a cellulose derived polymer, or combinations thereof, more preferably said anti-nucleating agent is selected from the group consisting of methacrylic acid-methyl methacrylate copolymer 1:1 (Eudragit L100), hydroxypropyl methylcellulose (HPMC) and HPMC acetate succinate (HPMC-AS), and combinations thereof.

### Embodiment 9

The pharmaceutical composition according to Embodiment 8, wherein the anti-nucleating agent is hydroxypropyl methylcellulose acetate succinate (HPMC-AS).

### Embodiment 10

The pharmaceutical composition according to any one of Embodiments 1 to 9, further comprising a filler, preferably the filler is selected from the group consisting of lactose, sucrose, glucose, mannitol, sorbitol, calcium phosphate, calcium carbonate, and cellulose or is a any combination said fillers, more preferably the filler is cellulose, even more preferably the filler is microcrystalline cellulose.

### Embodiment 11

The pharmaceutical composition according to any one of Embodiments 1 to 10, further comprising a disintegrant, preferably the filler is selected from the group consisting of starch, cellulose, cross-linked poly(N-vinylpyrrolidone), sodium starch glycolate, and sodium carboxymethyl cellulose or is a combination of two or more of said disintegrants, preferably the disintegrant is cross-linked poly(N-vinylpyrrolidone) or sodium carboxymethyl cellulose.

### Embodiment 12

The pharmaceutical composition according to any one of Embodiments 7 to 11, wherein said further components of the composition (i.e any one of the group selected from the anti-nucleating agent, the filler, the disintegrant, and combinations thereof, where present) are present in the extragranular phase.

### Embodiment 13

The pharmaceutical composition according to any one of Embodiments 1 to 12, wherein the pharmaceutical composition further comprises one or more of the following:
(a) a filler that is microcrystalline cellulose,
(b) a disintegrant that is crospovidone or sodium carboxymethyl cellulose,
(c) a lubricant that is magnesium stearate or sodium stearyl fumarate, and
(d) a glidant that is colloidal silicon dioxide.

### Embodiment 14

The pharmaceutical composition according to any one of Embodiments 7 to 13, wherein the hydroxypropyl methylcellulose acetate succinate (HPMC-AS) is present in about 5-15%, preferably in 7-10% by weight based on the total weight of the pharmaceutical composition (i.e., the weight of the granules and the extragranular ingredients together).

### Embodiment 15

The pharmaceutical composition according to any one of Embodiments 10 to 14, wherein the microcrystalline cellulose is present in about 30-85%, preferably in 40-70% by weight based on the total weight of the pharmaceutical composition (i.e., the weight of the granules and the extragranular ingredients together).

### Embodiment 16

The pharmaceutical composition according to any one of Embodiments 10 to 15, wherein the disintegrant is present in about 5-20%, preferably in 5-18%, more preferably in 10-15% by weight based on the total weight of the granules and all extragranular ingredients together.

### Embodiment 17

The pharmaceutical composition according to any one of Embodiments 1 to 16 comprising
(a) 5-15% by weight of granules which in turn comprise
   10-40% by weight of compound of formula (I);
   60-90% by weight of copovidone; and
   0.1-2% by weight colloidal silicon dioxide;
   based on the total weight of the granules;
(b) 50-85% by weight of microcrystalline cellulose;
(c) 5-15% by weight of crospovidone;
(d) 5-15%, by weight of HPMC acetate succinate;
(e) 1-5%, by weight of colloidal silicon dioxide; and
(f) 0.5-2%, by weight of magnesium stearate;
based on the total weight of the granules and all extragranular ingredients together.

### Embodiment 18

The pharmaceutical composition according to any one of Embodiments 1-17 comprising
(a) 15-70% by weight of granules which in turn comprise
   10-40% by weight of compound of formula (I);
   60-90% by weight of copovidone; and
   0.1-2% by weight colloidal silicon dioxide;
   based on the total weight of the granules;
(b) 20-75% by weight of microcrystalline cellulose;
(c) 5-15% by weight of crospovidone;
(d) 5-15% by weight of HPMC acetate succinate;
(e) 1-5% by weight of colloidal silicon dioxide; and
(f) 0.5-2% by weight of magnesium stearate;
based on the total weight of the granules and all extragranular ingredients together.

### Embodiment 19

The pharmaceutical composition according to any one of Embodiments 1-18 comprising
(a) 15-70% by weight of granules which in turn comprise
   10-40% by weight of the compound of formula (I);
   60-90% by weight of copovidone; and
   0.1-2% by weight colloidal silicon dioxide;
   based on the total weight of the granules;
(b) 20-75% by weight of microcrystalline cellulose;
(c) 5-15% by weight of sodium carboxymethyl cellulose;
(d) 5-15% by weight of HPMC acetate succinate;
(e) 1-5% by weight of colloidal silicon dioxide; and
(f) 0.5-2% by weight of magnesium stearate;
based on the total weight of the granules and all extragranular ingredients together.

### Embodiment 20

The pharmaceutical composition according to any one of Embodiments 1-19 comprising
(a) 15-70% by weight of granules which in turn comprise
   10-40% by weight of the compound of formula (I);
   60-90% by weight of copovidone; and
   0.1-2% by weight colloidal silicon dioxide;
   based on the total weight of the granules;
(b) 20-75% by weight of microcrystalline cellulose;
(c) 5-18% by weight of sodium carboxymethyl cellulose;
(d) 5-15% by weight of HPMC acetate succinate;
(e) 1-5% by weight of colloidal silicon dioxide; and
(f) 0.5-2% by weight of magnesium stearate;
based on the total weight of the granules and all extragranular ingredients together.

### Embodiment 21

The pharmaceutical composition according to any one of Embodiments 1-6 comprising
(a) 15-70% by weight of granules which in turn comprise
   10-40% by weight of the compound of formula (I);
   60-90% by weight of copovidone; and
   0.1-2% by weight colloidal silicon dioxide;
   based on the total weight of the granules;
(b) 20-75% by weight of microcrystalline cellulose;
(c) 5-15% by weight of crospovidone;
(d) 1-5% by weight of colloidal silicon dioxide; and
(e) 0.5-2% by weight of sodium stearyl fumarate;
based on the total weight of the granules and all extragranular ingredients together.

### Embodiment 22

The pharmaceutical composition according to any one of Embodiment 1 to 21, wherein the composition is in the form of a capsule, tablet, or sachet.

### Embodiment 23

A process for making the pharmaceutical composition as defined by any one of Embodiment 1 to 22 comprising a melt granulation or a melt extrusion step.

### Embodiment 24

The process according to Embodiment 23 further characterized by the following process steps:
(1) melt-granulating or melt-extruding, preferably melt-extruding the compound (4S,5R)-3-(2′-Amino-2-morpholin-4-yl-4′-trifluoromethyl-[4,5]bipyrimidinyl-6-yl)-4-hydroxymethyl-5-methyl-oxazolidin-2-one or any pharmaceutically acceptable salt thereof together with the polymer poly(N-vinylpyrrolidone) (PVP) or any derivative thereof, preferably, said polymer is a copolymer of N-vinylpyrrolidone and vinylacetate, and optionally colloidal silica;
(2) milling and/or screening the melt granulates obtained by step (1) to obtain melt granulates with a median particle size median particle size within 250 to 1000 μm, preferably within 300 to 750 μm, more preferably within 300 to 500 μm;
(3) blending the melt granulates obtained by step (2) with an anti-nucleating agent, preferably said anti-nucleating agent is an acrylic polymer or a cellulose derived polymer or combinations thereof, more preferably said anti-nucleating agent is selected from the group consisting of Eudragit L100, HPMC and HPMC-AS, even more preferably said anti-nucleating agent is hydroxypropyl methylcellulose acetate succinate (HPMC-AS), and optionally microcrystalline cellulose, and optionally a disintegrant, preferably, cross-linked poly(N-vinylpyrrolidone) or sodium carboxymethyl cellulose, and optionally a glidant, and optionally a lubricant;
(4) encapsulating or tabletting the blend obtained by step (3), preferably encapsulating said blend into hard gelatine capsules.

### Embodiment 25

The process according to Embodiment 23 further characterized by the following process steps:
(1) melt-granulating or melt-extruding, preferably melt-extruding the compound (4S,5R)-3-(2′-Amino-2-morpholin-4-yl-4′-trifluoromethyl-[4,5]bipyrimidinyl-6-yl)-4-hydroxymethyl-5-methyl-oxazolidin-2-one or any pharmaceutically acceptable salt thereof together with the polymer poly(N-vinylpyrrolidone) (PVP) or any derivative thereof, preferably, said polymer is a copolymer of N-vinylpyrrolidone and vinylacetate, and optionally colloidal silica;
(2) milling and/or screening the melt granulates obtained by step (1) to obtain melt granulates with a median particle size median particle size within 250 to 1000 μm, preferably within 300 to 750 μm;
(3) optionally blending the melt granulates obtained by step (2) with one or more excipients, including but not limited to microcrystalline cellulose, a disintegrant, preferably, crospovidone; a glidant, a lubricant, or a combination thereof; and
(4) encapsulating or tabletting the blend obtained by step (3), preferably tableting said blend into tablets.

### Embodiment 26

A pharmaceutical composition obtainable by the process according to any one of Embodiments 23-25.

### Embodiment 27

A pharmaceutical composition according to any one of Embodiments 1-22 for use in the treatment of cancer, for use in the treatment or suppression of tumors, or for use in the treatment or prevention of other conditions, diseases or disorders dependent on PI3K.

### EXAMPLES

Hereinafter, the present invention is described in more details and specifically with reference to the examples, which however are not intended to limit the present invention.

All the inactive ingredients (also referred to as excipients) referred herein are used in qualities suitable for pharmaceutical use and are commercially available under various brand names as indicated in the following as examples:

Methacrylic acid-methyl methacrylate copolymer 1:1, e.g. Eudragit L100
Copovidone, USP/NF, e.g. Kollidon VA 64
Colloidal silicon dioxide, USP/NF, e.g. Aerosil 200
Microcrystalline cellulose (Cellulose MK GR), USP/NF, e.g. Vivapur 102, Avicel PH 102
Microcrystalline cellulose (Microcrystalline cellulose powder), USP/NF, e.g. Vivapur 101, Avicel PH 101
Sodium carboxymethylcellulose, e.g., croscarmellose sodium (e.g., Ac-Di-Sol)
Crospovidone, USP/NF, e.g. Polyplasdone XL
Hypromellose acetate succinate, USP/NF, e.g. HPMC-AS, LF
Magnesium stearate, USP/NF
Povidone, USP/NF e.g. Kollidon K12
PEG-40 Hydrogenated castor oil, e.g. Cremophor RH 40 or Kolliphor RH 40

### Abbreviations:

ca.: about
HMPC: hydroxypropyl methylcellulose

### HPLC: High Performance Liquid Chromatography

min: minute
NMT: not more than
rpm: revolutions per minute

### USP/NF: US Pharmacopeia and National Formulary

### XRPD: X-ray Powder Diffraction

### Analytical Methods:

### Assay, Degradation Products/Impurities by HPLC

Principle: RP HPLC with UV detection

### Reagents:

Acetonitrile (HPLC grade, e.g. Merck)
Water (e.g. from Milli-Q instrument)
Methanol (Analytical grade, e.g. Sigma-Aldrich or equivalent)
Solvent: Water/acetonitrile 40/60 (v/v)

### Equipment:

Apparatus: e.g. HP 1290 (UPLC)
Column: Waters Acquity BEH C18 (Length 100 mm, internal diameter 2.1 mm or equivalent column and particle size 1.7 μm)

### Chromatographic Conditions

Mobile Phase:
A: Water/Acetonitrile/TFA (95:5:0.05 v/v/v)
B: Water/Acetonitrile/TFA (5:95:0.05 v/v/v)

### Gradient:

| Time [min.] | Phase A [%] | Phase B [%] | Curve |
|---|---|---|---|
| 0.0 | 100 | 0 | — |
| 4.5 | 75 | 25 | linear |
| 7.5 | 75 | 25 | linear |
| 16 | 0 | 100 | linear |
| 16.1 | 100 | 0 | linear |
| 18 | 100 | 0 | — |

Flow rate: 0.7 ml/min

### Detection: 248 nm

Column temperature: 30° C.
Auto-sampler temperature: Ambient
Needle wash: Methanol/water 80/20 (v/v)
Injection volume:
2 μl of the test and reference solutions, equivalent to about
0.6 μg of drug substance compound of formula (I) in the reference solution
   Run time: 18 min

For all calculations of degradation products disregard peaks <0.1% (reporting limit).

### Dissolution Test

Dissolution medium: 0.1 N HCl

### Dissolution Volume: 900 mL

USP apparatus: II

### RPM: 75

Sinker: Spring style capsule sinker

### Method: 248 nm UV by HPLC

Samples per test: 6 capsules

### Time Point: 15, 30, 45, 60 min

### Vessel: Regular

Dissolution System: Auto sampler

### Kinetic Solubility

Principle: Measurement of the amount of drug substance solubilized using rotating bottle apparatus. Determination by HPLC/UV method for sample analysis.

### Preparation of Test Medium:

### Kinetic Solubility Conditions:

Speed of rotation: 50 rpm
Test medium: 0.01 N HCl
Volume of test medium: 75 mL
Temperature: 37±0.5° C.
   Sampling Time points 5, 10, 15, 20, 25, 30, 45, 60 minutes

### Sample Size 400 to 500 mg

Procedure: The concentration maximum is taken as kinetic solubility value.

### X-Ray Powder Diffraction (XRPD) Analysis

Principle: X-ray powder diffraction (XRPD) in reflection geometry
Equipment: Powder diffractomer e.g. type Bruker D8 Discover
Procedure: Place the test substance on the specimen holder. Record the X-ray diffraction pattern between 5° and 40° (2-theta) with Cu Kα radiation (1.5406 Å) in reflection geometry using the following conditions
Detector type: Lynx Eye
High voltage: 40 kV
Current: 40 mA
Scan type: continuous
Step time: 0.3 s
Step size: 0.016°
Soller slit: 2.5°
Slits: 0.6 mm
Method: A part of the sample is used without preparation for the measurement. The sample carrier is filled completely with the test substance.
   Place the sample on the specimen holder. Record the X-ray diffraction pattern between 5° and 40° (2Theta) with CuKα radiation.
Evaluation: Observe the recorded diffractogram and report any detectable diffraction peak. A peak is said to be “detectable” if the signal to noise ratio is greater than 3. Diffraction peaks are sharp (generally <2° (2-theta) at the base)
Assessment: The sample is amorphous if no detectable diffraction peak has to be reported. Very broad peak(s) characteristic of amorphous material may be observed (halo of diffusion).

### Example 1: Manufacturing Process for 2.5, 10, and 50 mg Dosage Strength

In the following, the manufacturing process is outlined for the granules and capsules in all exemplified dosage strengths. The corresponding amounts of the ingredients are provided in Tables 1.1, 1.2, 1.3, and 1.4 below.

### Manufacturing of the Granules (Melt Extrudates):

The appropriate amounts of copovidone, colloidal silicon dioxide, and the compound of formula (I) are weighed out. Said ingredients are blended at 20 rpm for 5 min in a Bohle (or TOTE) bin blender. The blend is passed through a 25-30 mesh screen or a Comil screen of type 024R03125 with impeller and then blended again for 15 min at 20 rpm using the Bohle bin blender. The resulting blend is hot melt extruded on an 18 mm Leistritz horizontal screw extruder at process temperatures from 100° C. over 150° C. to finally 200° C. (low shear screw design, about 100 rpm screw speed, about 15±3 g/min feed rate).

### Manufacturing of the Blend:

The appropriate amount of melt extrudate, obtained above, is milled using a Fitz mill (screen type 1512-0020 for 2.5 mg and 1512-0033 for 10 and 50 mg dosage strength) in a hammer (impact mill, hammer forward setting at 5500±100 rpm for 2.5 mg) and knife (forward setting 2500±100 rpm for 10 mg and 1800-2000 rpm for 50 mg). In case of 2.5 mg dosage strength, the milled extrudate is screened through a 100 mesh sieve as an additional process step. Appropriate amounts of the milled extrudate, the microcrystalline cellulose, crospovidone, hypromellose acetate succinate LF, and colloidal silicon dioxide are weighed out and blended for 10 min at 20 rpm using a Bohle bin blender. The resulting blend is then passed through an 18 mesh screen or a Comil screen of type 039R03125. The screened blend is blended a second time for 10 min at 20 rpm and screened again through an 18 mesh blend or a Comil screen of type 039R03125. Appropriate amounts of magnesium stearate are weighed out and screened through a 35 mesh sieve. The screened magnesium stearate is blended together with the other blend materials for 3 min at 20 rpm using the Bohle bin blender to give the final blend which is then filled into capsules.

### Manufacturing of the Capsules:

The final blend is then filled into capsules of appropriate size by using dosing disk or dosator encapsulation machines (e.g. Bosch or Zanasi). The capsules are stored not above 25° C. under protection from moisture.

TABLE 1.1
Composition of granules (melt extrudates)

| | Amount | Quantity per batch |
|---|---|---|
| Component | [weight %] | [g/batch] |
| Compound of Formula I | 35.00 | 7700 |
| Kollidon VA64 (USP/NF) | 64.50 | 14190 |
| Aerosil 200PH (USP/NF) | 0.50 | 110 |
| Total weight | 100.0 | 22000 |

TABLE 1.2
Composition of capsules with 2.5 mg dosage strength

| | Amount | Quantity | Quantity |
|---|---|---|---|
| | per capsule | per capsule | per batch |
| Component | [weight %] | [mg/capsule] | [g/batch] |
| Granules (see Table 1.1) | 9.52 | 7.14 | 571.20 |
| Cellulose MK GR (USP/NF) | 66.48 | 49.86 | 3988.80 |
| Crospovidone (USP/NF) | 10.00 | 7.50 | 600.00 |
| HPMC-AS (LF) (USP/NF) | 10.00 | 7.50 | 600.00 |
| Aerosil 200PH (USP/NF) | 3.00 | 2.25 | 180.00 |
| Magnesium stearate PH | 1.00 | 0.75 | 60.00 |
| (USP/NF) | | | |
| Total final blend | 100.00 | 75 | 6000.00 |
| Hard gelatin capsule, size 3 | | 48 | 3840.00 |
| Total capsule weight | | 123.00 | 9840.00 |

TABLE 1.3
Composition of capsules with 10 mg dosage strength

| | Amount | Quantity | Quantity |
|---|---|---|---|
| | per capsule | per capsule | per batch |
| Component | [weight %] | [mg/capsule] | [g/batch] |
| Granules (see Table 1.1) | 35.71 | 28.57 | 2571.30 |
| Cellulose MK GR (USP/NF) | 40.29 | 32.23 | 2900.70 |
| Crospovidone (USP/NF) | 10.00 | 8.00 | 720.00 |
| HPMC-AS (LF) (USP/NF) | 10.00 | 8.00 | 720.00 |
| Aerosil 200PH (USP/NF) | 3.00 | 2.40 | 216.00 |
| Magnesium stearate PH | 1.00 | 0.80 | 72.00 |
| (USP/NF) | | | |
| Total final blend | 100.00 | 80 | 7200.00 |
| Hard gelatin capsule, size 1 | | 76 | 6840.00 |
| Total capsule weight | | 156.00 | 14040.00 |

TABLE 1.4
Amount of capsules with 50 mg dosage strength

| | Amount | Quantity | Quantity |
|---|---|---|---|
| | per capsule | per capsule | per batch |
| Component | [weight %] | [mg/capsule] | [g/batch] |
| Granules (see Table 1.1) | 35.71 | 142.85 | 17142.40 |
| Cellulose MK GR (USP/NF) | 40.29 | 161.15 | 19338.60 |
| Crospovidone (USP/NF) | 10.00 | 40.00 | 4800.00 |
| HPMC-AS (LF) (USP/NF) | 10.00 | 40.00 | 4800.00 |
| Aerosil 200PH (USP/NF) | 3.00 | 12.00 | 1440.00 |
| Magnesium stearate PH | 1.00 | 4.00 | 480.00 |
| (USP/NF) | | | |
| Total final blend | 100.00 | 400.00 | 48000.00 |
| Hard gelatin capsule, size 00 | | 118.00 | 14160.00 |
| Total capsule weight | | 518.00 | 62160.00 |

### Example 2: Physicochemical Characterization of the Melt Extrudates

Chemical Compatibility of the Compound of Formula (I) with Polymers

The compatibility of the compound of formula (I) with various polymers was tested by preparing melt extrudates according to the manufacturing process as described in example 1 and analyzing the resulting melt extrudates by HPLC to determine the amount of degradation or related products (“impurities”) of the compound of formula (I). The product temperature range was kept in the range of 195-205° C. as the inventors of the present invention found that processing temperatures exceeding 205° C. led to the degradation of compound of formula (I). The amount of total impurities was assessed according to the guideline that the sum of specified and unspecified impurities (herein referred to as total impurity) is preferably not more than (NMT) 0.5%. Copovidone (Kollidon VA 64), povidone (Kollidon 12), HMPC, Cremophor RH 40 and combinations thereof were tested. The compositions tested and the results obtained are shown in Table 2.1.

For the composition comprising Cremophor RH 40 a total impurity of 0.728% w/w was found (not shown in Table 2.1). The total impurity level exceeded the acceptance limit of 0.5% in compositions comprising HPMC. However, for, the impurity levels were found to be well below 0.5% in compositions comprising only copovidone and/or povidone as polymer.

Compositions with copovidone as polymer alone are preferred since povidone (e.g. Kollidon K12) is highly hygroscopic. This may lead to potential chemical and physical stability issues when using povidone in the composition, which would require taking extra precautions to prevent the absorption of water when formulating the compositions.

TABLE 2.1
Chemical compatibility

| | Composition of melt extrudates | | | | | Process | |
|---|---|---|---|---|---|---|---|
| | Compound | Copovidone | Povidone | | Colloidal | Product | |
| | of formula | (Kollidon | (Kollidon | | silicon | Temp. | HPLC |
| Batch | (I) | VA 64) | K12) | HPMC | dioxide | Range | % Total |
| ID | (% w/w) | (% w/w) | (% w/w) | (% w/w) | (% w/w) | (° C.) | Impurity |
| A | 35.00 | 64.50 | — | — | 0.50 | 199-204 | 0.313 |
| B | 35.00 | 54.83 | 9.67 | — | 0.50 | 195-206 | 0.283 |
| C | 35.00 | 46.00 | 18.50 | — | 0.50 | 195-201 | 0.313 |
| D | 35.00 | 32.50 | 32.00 | — | 0.50 | 198-200 | 0.264 |
| E | 35.00 | 43.75 | 18.75 | 2.00 | 0.50 | 195-204 | 0.536 |
| F | 35.00 | 50.57 | 8.93 | 5.00 | 0.50 | 200-203 | 0.601 |

### Thermoanalysis of the Melt Extrudates

Melt extrudates with compositions as indicated in Table 2.2 were prepared according to the manufacturing process as described in example 1 and their melting point and glass transition temperatures (T_{g}) were determined.

Compositions comprising HPMC were found to have higher melting points than compostions comprising copovidone or povidone alone or combinations thereof.

As the present inventors found that processing temperatures exceeding 205° C. lead to the degradation of the compound of formula (I), compositions with HPMC with their increased melting points were found to be less suitable.

TABLE 2.2
Melting points of polymer mixtures

| | Composition of melt extrudates | | | | | Melting Point | | |
|---|---|---|---|---|---|---|---|---|
| | Compound | Copovidone | Povidone | | Colloidal | Data (OptiMelt) | | |
| | of formula | (Kollidon | (Kollidon | | silicon | Onset | Single | DSC |
| Batch | (I) | VA64) | K12) | HPMC | dioxide | Point | Point | T_{g} |
| ID | (% w/w) | (% w/w) | (% w/w) | (% w/w) | (% w/w) | (° C.) | (° C.) | (° C.) |
| G | 30.00 | 69.50 | — | — | 0.50 | 167 | 180 | 117.0 |
| H | 30.00 | — | 34.50 | 34.75 | 0.50 | 202 | 211 | 124.0 |
| I | 30.00 | — | 69.50 | — | 0.50 | 173 | 181 | ND |
| J | 30.00 | 49.50 | 20.00 | — | 0.50 | 175 | 200 | 116.5 |
| L | 30.00 | 49.50 | — | 20.00 | 0.50 | 194 | 209 | 119.6 |
| M | 30.00 | 39.50 | 20.00 | 10.00 | 0.50 | 182 | 201 | 117.6 |
| N | 35.00 | 64.50 | — | — | 0.50 | 172 | 183 | ca. 115 |
| O | 35.00 | 44.50 | 20.00 | — | 0.50 | 178 | 188 | 117.7 |
| P | 35.00 | 34.50 | 20.00 | 10.00 | 0.50 | 180 | 199 | ca. 115 |

ND: Not determined.

### Solubility and Physical Stability of Melt Extrudates

Melt extrudates were prepared according to example 1. The kinetic solubility and the physical state of the compound of formula (I) were determined. Melt extrudates of different compositions were tested and the results are displayed in Table 2.3. Kinetic solubility were determined by adding melt extrudates equivalent to 100 mg of the compound of formula (I) in pH 6.8 phosphate buffer using a rotating bottle at 50 rpm and at a temperature of 37° C. The physical state (crystalline or amorphous) was determined by XRPD (discrete peaks indicating some of the material being in a crystalline state, absence of peaks indicating amorphous state).

As shown below, compositions with copovidone alone stabilized the compound in its amorphous form up to a drug load of 35%. At 40% drug load the compound of formula (I) crystallized out in the copovidone polymer matrix. The addition of povidone to compostions containing copovidone stabilized the amorphous state of the compound of formula (I) but at the same time a decrease in kinetic solubility was observed. Therefore, a drug load of 35% in copovidone alone is preferred in the light of both solubility and stability requirements.

TABLE 2.3
Solubility and physical stability

| | Composition of melt extrudates | | | | | |
|---|---|---|---|---|---|---|
| | Compound | Copovidone | Povidone | Colloidal | | |
| | of formula | (Kollidon | (Kollidon | silicon | Kinetic | Physical state of |
| Batch | (I) | VA 64) | K12) | dioxide | Solubility | compound of formula |
| ID | (% w/w) | (% w/w) | (% w/w) | (% w/w) | (mg/mL) | (I) (XRPD) |
| Q | 100 | — | — | — | ca. 0.04 | Crystalline |
| R | 30.00 | 69.50 | — | 0.50 | 0.53 | Amorphous |
| S | 35.00 | 64.50 | — | 0.50 | 0.48 | Amorphous |
| T | 40.00 | 59.50 | — | 0.50 | ND | Crystalline |
| U | 40.00 | 50.60 | 8.90 | 0.50 | ND | Crystalline |
| V | 40.00 | 42.40 | 17.10 | 0.50 | 0.43 | Amorphous |
| W | 40.00 | — | 59.50 | 0.50 | ND | Amorphous |

ND: Not determined.

### Example 3: Particle Size Distribution of Milled Extrudate and its Influence on Gelation and Dissolution

Melt extrudate with 35% drug load of compound of formula (I), 54.5% copovidone, 0.5% colloidal silicon dioxide were prepared according to the process as described in example 1. The melt extrudates were milled and the resulting particle size distributions were determined by sieve analysis, e.g. by giving ca. 10 g sample on the sieve stack comprising 6 to 8 sieves with apertures within the range of 50-1000 micron (μm) which is shaken repeatively for ca. 5 minutes time intervals until less than 0.2% of the material passes a given sieve aperture in any of said 5 minutes intervals). The detailed particle size distributions for melt extrudates milled with three different speeds are provided in Tables 3.1 to 3.3. When those milled melt extrudates were further processed to capsules according to the process as described in example 1 and the capsules exposed to aqueous media, an undesired gel formation was observed for melt extrudates with a median particle size below 300 micron. This caused a reduction in the disintegration time of the capsules and a reduction of the dissolution rate of the drug substance. For melt extrudates with a median particle size above 300 micron no gel formation was observed and the capsule could disintegrate quickly and the drug substance could dissolve quickly and completely.

Table 3.4 further elucidates this observed effect by providing in vitro dissolution data. The undesired gelling effect was observed in capsules made with extrudates with a median particle size of ca. 232 micron, obtained e.g. after milling at 2500 rpm, Consequently this resulted in a slower dissolution rate in 0.1 N HCl. Drug release was only 61% and highly variable (relative standard deviation (RSD): 36.9%) after 15 min. Even after 60 min only 74% of the compound of formula (I) was released (RSD 29.8).

In contrast, using extrudates with a median particle size of ca. 384 micron, e.g obtained by milling at 2000 rpm, resulted in the absence of any gelling effect when the corresponding capsules were dissolved in 0.1 N HCl. The drug release was >90% after only 15 min and the relative standard deviation of the test data (n=6) was as low as 6.4%. The drug release can therefore be considered as fast and reliably consistent.

TABLE 3.1
Sieve analysis of melt extrudates milled in a Fitz mill
with a knife forward setting of 2500 rpm

| Sieve | Size [μm] | Mid size [μm] | Weight [%] |
|---|---|---|---|
| Upper Limit | 1500 | | |
| Sieve 1 | 1000 | 1250 | 1.1 |
| Sieve 2 | 707 | 853.5 | 2.2 |
| Sieve 3 | 500 | 603.5 | 6.0 |
| Sieve 4 | 400 | 450 | 5.5 |
| Sieve 5 | 297 | 348.5 | 20.8 |
| Sieve 6 | 250 | 273.5 | 13.0 |
| Pan | 0 | 125 | 54.1 |
| Total weight | | | 102.7 |
| Mid Points Mean: | | 262.0789 | |
| Linear Interpolated Median: | | 237.2921 | |

The melt extrudates of Table 3.1 were found to induce undesired gel formation.

TABLE 3.2
Sieve analysis of melt extrudates milled in a Fitz mill
with a knife forward setting of 1900 rpm

| Sieve | Size [μm] | Mid size [μm] | | Weight [%] |
|---|---|---|---|---|
| Upper Limit | 1500 | | | |
| Sieve 1 | 1000 | 1250 | | 1.0 |
| Sieve 2 | 840 | 920 | | 1.3 |
| Sieve 3 | 420 | 603 | | 58.6 |
| Sieve 4 | 250 | 335 | | 25.2 |
| Sieve 5 | 125 | 187.5 | | 13.8 |
| Sieve 6 | 53 | 89 | | 2.2 |
| Pan | 0 | 26.5 | | 2.3 |
| Total weight | | | | 104.4 |
| Mid Points Mean: | | 485.1557 | μm | |
| Linear Interpolated Median: | | 482.3549 | μm | |

The melt extrudates of Table 3.2 were found not to induce undesired gel formation.

TABLE 3.3
Melt extrudates milled in a Fitz mill with a knife forward
setting of 2000 rpm

| Sieve | Size [μm] | Mid size [μm] | | Weight [%] |
|---|---|---|---|---|
| Upper Limit | 1200 | | | |
| Sieve 1 | 1000 | 1100 | | 0 |
| Sieve 2 | 850 | 925 | | 0.782857 |
| Sieve 3 | 425 | 637.5 | | 33.72918 |
| Sieve 4 | 250 | 337.5 | | 39.83281 |
| Sieve 5 | 180 | 215 | | 10.64154 |
| Sieve 6 | 125 | 152.5 | | 6.136801 |
| Sieve 7 | 75 | 100 | | 5.612685 |
| Sieve 8 | 63 | 69 | | 1.214091 |
| Pan | 0 | 31.5 | | 2.050023 |
| Total weight | | | | 100.0 |
| Mid Points Mean: | | 396.0348 | μm | |
| Linear Interpolated Median: | | 356.9558 | μm | |

The melt extrudates of Table 3.3 were found not to induce undesired gel formation.

TABLE 3.4
Dissolution test in 0.1N HCl, USP II, 75 rpm of 50 mg dosage
strength capsules prepared with extrudate of different particle
sizes; mean, minimum, and maximum values of 6 capsules per
test (n = 6) and relative standard deviation.

| | | Dissolution time [min] | | | |
|---|---|---|---|---|---|
| | | 15 | 30 | 45 | 60 |
| Extrudate milled at 2500 rpm | Mean [%] | 61 | 68 | 71 | 74 |
| Median particle size: 232 micron | Min. [%] | 44 | 50 | 55 | 58 |
| Gelling issue: reduced and | Max. [%] | 89 | 100 | 101 | 102 |
| highly variable drug release rate | RSD [%] | 36.9 | 35.3 | 32.3 | 29.8 |
| Extrudate milled at 1800 rpm | Mean | 92 | 98 | 99 | 100 |
| Median particle size: 384 micron | Min. | 84 | 89 | 90 | 90 |
| No gelling issue: fast and | Max. | 99 | 103 | 104 | 104 |
| reliable drug release | % RSD | 6.4 | 5.2 | 5.2 | 5.3 |

### Example 4: Effect of Anti-Nucleating Agent

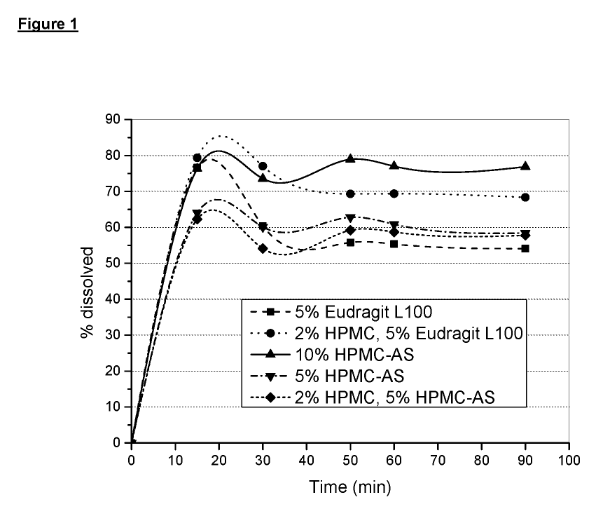

Capsules were prepared according to the process as described with in example 1 but with different types and amounts of external stabilizers. If the amounts are less than 10%, an increased amount of cellulose as filler was used. As external stabilizers Eudragit L100, HPMC, HPMC-AS, and combinations thereof have been prepared and analyzed by an in vitro dissolution test with using 0.01 N HCl during the acid stage (0, 15, 30 min timepoints) and pH 6.8 sodium phosphate buffer for the buffer stage (50, 60, 90 min timepoints) The results are shown in FIG. 1. The composition with 10% HPMC-AS exhibited the most preferred dissolution pattern.

### Example 5: Manufacturing Process for 25 mg Dosage Strength

In the following, the manufacturing process is outlined for the granules and capsules in all exemplified dosage strengths. The corresponding amounts of the ingredients are provided in Tables 5.1 and 5.2 below.

### Manufacturing of the Granules (Melt Extrudates):

The appropriate amounts of copovidone, colloidal silicon dioxide, and the compound of formula (I) are weighed out. Said ingredients are blended at 20 rpm for 5 min in a Bohle (or TOTE) bin blender. The blend is passed through a 25-30 mesh screen or a Comil screen of type 024R03125 with impeller and then blended again for 15 min at 20 rpm using the Bohle (or TOTE) bin blender. The resulting blend is hot melt extruded on an 18 mm Leistritz horizontal screw extruder at process temperatures from 100° C. over 150° C. to finally 200° C. (low shear screw design, about 100 rpm screw speed, about 15±3 g/min feed rate).

### Manufacturing of the Blend:

The appropriate amount of melt extrudate, obtained above, is milled using a Fitz mill (screen type 1512-0033) in a knife (forward setting 1900±100 rpm). The milled extrudate is screened through a 44 micron (mesh #325) screen, and the material retained above the mesh is collected for encapsulation.

Appropriate amounts of the retained milled extrudate is weighed out and passed along with colloidal silicon dioxide through an 18 mesh screen. The screened material is blended for 5 minutes at 20 rpm using a Bohl (or TOTE) bin blender. Appropriate amounts of the microcrystalline cellulose, sodium carboxymethylcellulose, and hypromellose acetate succinate LF are weighed out and blended with the prior screened blend for 10 minutes at 20 rpm using a Bohle (or TOTE) bin blender. The resulting blend is then passed through an 18 mesh screen or Comil screen of type 039R03125. The screened material is blended a second time for 10 minutes at 20 rpm using Bohle (or TOTE) bin blender and screened again through an 18 mesh screen or Comil screen of type 039R03125. Appropriate amounts of magnesium stearate are weighed out and screened through a 35 mesh sieve. The screened magnesium stearate is blended together with the other blend materials for 3 min at 20 rpm using the Bohle (or TOTE) bin blender to give the final blend which is then filled into capsules.

### Manufacturing of the Capsules:

The final blend is then filled into capsules of appropriate size by using dosing disk or dosator encapsulation machines (e.g. Bosch or Zanasi). The capsules are stored not above 25° C. under protection from moisture.

TABLE 5.1
Composition of the granules (melt extrudates)

| | Amount | Quantity per batch |
|---|---|---|
| Component | [weight %] | [g/batch] |
| Compound of Formula I | 35.00 | 7700 |
| Kollidon VA64 (USP/NF) | 64.50 | 14190 |
| Aerosil 200PH (USP/NF) | 0.50 | 110 |
| Total weight | 100.0 | 22000 |

TABLE 5.2
Composition of capsules with 25 mg dosage strength

| | Amount | Quantity | Quantity |
|---|---|---|---|
| | per capsule | per capsule | per batch |
| Component | [weight %] | [mg/capsule] | [g/batch] |
| Granules (see Table 5.1) | 22.32 | 71.43 | 10714.5 |
| Cellulose MK GR (USP/NF), | 28.60 | 91.54 | 13731.0 |
| Avicel PH-102 | | | |
| Microcrystalline Cellulose | 28.60 | 91.53 | 13729.5 |
| powder (USP/NF), Avicel | | | |
| PH-101 | | | |
| Croscarmellose Sodium | 10.16 | 32.5 | 4875.0 |
| (USP/NF) | | | |
| HPMC-AS (LF) (USP/NF) | 7.81 | 25.0 | 3750.0 |
| Aerosil 200PH (USP/NF) | 1.88 | 6.00 | 900.0 |
| Magnesium stearate PH | 0.63 | 2.0 | 300.0 |
| (USP/NF) | | | |
| Total final blend | 100.00 | 320.00 | 48000.0 |
| Hard gelatin capsule, size 0 | | 96.00 | 14400.00 |
| Total capsule weight | | 416.00 | 62400.00 |

### Example 6: Manufacturing Process for 40 mg Dosage Strength

In the following, the manufacturing process is outlined for the granules and capsules in all exemplified dosage strengths. The corresponding amounts of the ingredients are provided in Tables 6.1 and 6.2 below.

### Manufacturing of the Granules (Melt Extrudates):

The appropriate amounts of copovidone, colloidal silicon dioxide, and the compound of formula (I) are weighed out. Said ingredients are blended at 20 rpm for 5 min in a Bohle (or TOTE) bin blender. The blend is passed through a 25-30 mesh screen or a Comil screen of type 024R03125 with impeller and then blended again for 15 min at 20 rpm using the Bohle (or TOTE) bin blender. The resulting blend is hot melt extruded on an 18 mm Leistritz horizontal screw extruder at process temperatures from 100° C. over 150° C. to finally 200° C. (low shear screw design, about 100 rpm screw speed, about 15±3 g/min feed rate).

### Manufacturing of the Blend:

The appropriate amount of melt extrudate, obtained above, is milled using a Fitz mill (screen type 1512-0033) in a knife (forward setting 1900±100 rpm). The milled extrudate is screened through a 88 micron (mesh #170) screen, and the material retained above the mesh is collected for encapsulation.

The appropriate amount of the retained milled extrudate is weighed out and passed along with colloidal silicon dioxide through an 18 mesh screen. The screened material is blended for 5 minutes at 20 rpm using a Bohl (or TOTE) bin blender. Appropriate amounts of the Cellulose MK GR (Avicel PH-101) is weighed out and blended with the prior screened blend for 5 minutes at 20 rpm using a Bohl (or TOTE) bin blender. Appropriate amounts of the microcrystalline cellulose, sodium carboxymethylcellulose, and hypromellose acetate succinate LF are weighed out and blended with the prior screened blend for 10 minutes at 20 rpm using a Bohle (or TOTE) bin blender. The resulting blend is then passed through an 18 mesh screen or Comil screen of type 039R03125. The screened material is blended a second time for 10 minutes at 20 rpm using Bohle (or TOTE) bin blender and screened again through an 18 mesh screen or Comil screen of type 039R03125. Appropriate amounts of magnesium stearate are weighed out and screened through a 35 mesh sieve. The screened magnesium stearate is blended together with the other blend materials for 3 min at 20 rpm using the Bohle (or TOTE) bin blender to give the final blend which is then filled into capsules.

### Manufacturing of the Capsules:

The final blend is then filled into capsules of appropriate size by using dosing disk or dosator encapsulation machines (e.g. Bosch or Zanasi). The capsules are stored not above 25° C. under protection from moisture.

TABLE 6.1
Composition of the granules (melt extrudates)

| | Amount | Quantity per batch |
|---|---|---|
| Component | [weight %] | [g/batch] |
| Compound of Formula I | 35.00 | 7700 |
| Kollidon VA64 (USP/NF) | 64.50 | 14190 |
| Aerosil 200PH (USP/NF) | 0.50 | 110 |
| Total weight | 100.0 | 22000 |

TABLE 6.2
Composition of capsules with 40 mg dosage strength

| | Amount | Quantity | Quantity |
|---|---|---|---|
| | per capsule | per capsule | per batch |
| Component | [weight %] | [mg/capsule] | [g/batch] |
| Granules (see Table 6.1) | 25.97 | 114.29 | 13713.6 |
| Cellulose MK GR (USP/NF), | 22.92 | 100.83 | 12099.6 |
| Avicel PH-102 | | | |
| Microcrystalline Cellulose | 22.91 | 100.80 | 12096.0 |
| powder (USP/NF), Avicel | | | |
| PH-101 | | | |
| Croscarmellose Sodium | 15.0 | 66.0 | 7920.0 |
| (USP/NF) | | | |
| HPMC-AS (LF) (USP/NF) | 10.0 | 44.0 | 5280.0 |
| Aerosil 200PH (USP/NF) | 2.40 | 10.56 | 1267.2 |
| Magnesium stearate PH | 0.80 | 3.52 | 422.4 |
| (USP/NF) | | | |
| Total final blend | 100.00 | 440.0 | 52798.8 |
| Hard gelatin capsule, size 00 | | 118.0 | 14160.0 |
| Total capsule weight | | 558.0 | 66958.8 |

### Example 7: Manufacturing Process for 50 mg Tablet

In the following, the manufacturing process is outlined for the granules and tablet in the exemplified dosage strength. The corresponding amounts of the ingredients are provided in Tables 7.1 and 7.2 below.

### Manufacturing of the Granules (Melt Extrudates):

The appropriate amounts of copovidone, colloidal silicon dioxide, and the compound of formula (I) are weighed out. Said ingredients are blended at 20 rpm for 5 min in a Bohle (or TOTE) bin blender. The blend is passed through a 30 mesh screen or a Comil screen mill (type U10: 7A039R03125 or S-197: 2A039R0325 024R03125) and then blended again for 15 min at 20 rpm using the Bohle (or TOTE) bin blender. The resulting blend is hot melt extruded on an 18 mm Leistritz horizontal screw extruder at process temperatures from 100° C. to 150° C. to finally 200° C. (low shear screw design, about 90-110 rpm screw speed, about 14±3 g/min feed rate).

### Manufacturing of the Blend:

The appropriate amount of melt extrudate, obtained above, is blended with colloidal silicon dioxide, for 5 min at 10 rpm in a Bohle bin blender. The blend is passed through a Frewitt screening mill with a 0.8 mm screen and with an oscillating bar. The appropriate amounts of the excipients for the internal phase are weighed out and added to the container containing the blend in the following order: crospovidone, sodium stearyl fumarate and cellulose MK GR. The mixture is blended for 5 minutes at 10 rpm in a Bohle bin blender. The resulting blend is passed through a Frewitt screening mill with a 0.8 mm screen and with an oscillating bar. The resulting mixture is further blended for 15 minutes at 10 rpm in a Bohle bin blender.

The final blend is then compresäsed into tablets of appropriate size by using a suitable rotary tablet press machine (e.g., FETTE 1200i TP09) fitted with 16×6.3 mm, R 3.5 punches. The tablets are prepared under controlled relative humidity of 30-40%. The tablets are dedusted using a suitable tablet deduster (e.g., Krämer). Tablets are stored not above 25° C. under protection from moisture.

TABLE 7.1
Composition of the granules (melt extrudates)

| | Amount | Quantity per batch |
|---|---|---|
| Component | [weight %] | [g/batch] |
| Compound of Formula I | 30.00 | 3.00 |
| Kollidon VA64 (USP/NF) | 69.50 | 6.95 |
| Aerosil 200PH (USP/NF) | 0.50 | 0.05 |
| Total weight | 100.0 | 10.00 |

TABLE 7.2
Composition of tablets with 50 mg dosage strength

| | Amount | Quantity | Quantity |
|---|---|---|---|
| | per capsule | per capsule | per 10,000 units |
| Component | [weight %] | [mg/capsule] | [g] |
| Granules (see Table 7.1) | 34.72 | 166.67 | 1666.7 |
| Cellulose MK GR | 52.53 | 252.13 | 2521.3 |
| Crospovidone | 10.00 | 48.00 | 480.0 |
| Aerosil 200 PH | 1.50 | 7.20 | 72.0 |
| Sodium stearyl fumarate | 1.25 | 6.00 | 60.0 |
| Total final blend | 100.00 | 480.00 | 4800.00 |

The in-process controls are as follows (target values):

| Control | Range |
|---|---|
| Shape | Ovaloid, 16.0 × 6.3 mm |
| Tablet thickness | 5.8 mm |
| Hardness | 200 N target, range of 140-260 N |
| Friability | ≧6.5 g, 0 breakage, ≦0.8% Abrasian |
| Disintegration time (without disk): | ≦10 min |
| 6 units, purified water, | |
| 37° C. ± 0.5° C. | |

1. A pharmaceutical composition comprising the compound (4S,5R)-3-(2′-Amino-2-morpholin-4-yl-4′-trifluoromethyl-[4,5]bipyrimidinyl-6-yl)-4-hydroxymethyl-5-methyl-oxazolidin-2-one (“compound of formula (I)”) or a pharmaceutically acceptable salt thereof, and a stabilizing polymer.

2. The pharmaceutical composition according to claim 1, wherein the stabilizing polymer is poly(N-vinylpyrrolidone) (PVP) or a derivative thereof, preferably, said polymer is a copolymer of N-vinylpyrrolidone and vinylacetate.

3. The pharmaceutical composition according to claim 1, wherein the compound of formula (I) or a pharmaceutically acceptable salt thereof, and the stabilizing polymer are present in the form of granules.

4. The pharmaceutical composition according to claim 3, wherein the granules comprise about 5-50% by weight of the compound of formula (I) based on the total weight of said granules.

5. The pharmaceutical composition according to claim 3, wherein the granules have a median particle size ranging from 250 to 1000 μm.

6. The pharmaceutical composition according to claim 1, further comprising an anti-nucleating agent.

7. The pharmaceutical composition according to claim 6, wherein the anti-nucleating agent is selected from the group consisting of methacrylic acid-methyl methacrylate copolymer 1:1 (Eudragit L100), hydroxypropyl methylcellulose (HPMC) and HPMC acetate succinate (HPMC-AS), and combinations thereof.

8. The pharmaceutical composition according to claim 6, wherein the anti-nucleating agent is hydroxypropyl methylcellulose acetate succinate (HPMC-AS).

9. The pharmaceutical composition according to any one of claim 1, further comprising a filler, a disintegrant, a lubricant, a glidant, or a combination thereof.

10. The pharmaceutical composition according to claim 9, wherein the pharmaceutical composition further comprises one or more of the following:
(a) a filler that is microcrystalline cellulose,
(b) a disintegrant that is crospovidone or sodium carboxymethyl cellulose,
(c) a lubricant that is magnesium stearate or sodium stearyl fumarate, and
(d) a glidant that is colloidal silicon dioxide.

11. The pharmaceutical composition according to claim 6, wherein said further components of the composition are present in the extragranular phase.

12. The pharmaceutical composition according to claim 6, wherein the hydroxypropyl methylcellulose acetate succinate (HPMC-AS) is present in about 5-15% by weight based on the total weight of granules and all extragranular ingredients together.

13. The pharmaceutical composition according to claim 9, wherein the microcrystalline cellulose is present in about 30-85% by weight based on the total weight of granules and all extragranular ingredients together.

14. The pharmaceutical composition according to claim 9, wherein the disintegrant is present in about 5-20% by weight based on the total weight of granules and all extragranular ingredients together.

15. The pharmaceutical composition according to claim 1, comprising
(a) 5-15% by weight of granules which in turn comprise
10-40% by weight of compound (4S,5R)-3-(2′-Amino-2-morpholin-4-yl-4′-trifluoromethyl-[4,5]bipyrimidinyl-6-yl)-4-hydroxymethyl-5-methyl-oxazolidin-2-one;
60-90% by weight of copovidone; and
0.1-2% by weight colloidal silicon dioxide;
based on the total weight of the granules;
(b) 50-85% by weight of microcrystalline cellulose;
(c) 5-15% by weight of crospovidone;
(d) 5-15%, by weight of HPMC acetate succinate;
(e) 1-5%, by weight of colloidal silicon dioxide; and
(f) 0.5-2%, by weight of magnesium stearate;
based on the total weight of granules and all extragranular ingredients together.

16. The pharmaceutical composition according to claim 1, comprising
(a) 15-70% by weight of granules which in turn comprise
10-40%, by weight of compound of formula (I);
60-90% by weight of copovidone; and
0.1-2% by weight colloidal silicon dioxide;
based on the total weight of the granules;
(b) 20-75% by weight of microcrystalline cellulose;
(c) 5-15% by weight of crospovidone;
(d) 5-15% by weight of HPMC acetate succinate;
(e) 1-5% by weight of colloidal silicon dioxide; and
(f) 0.5-2% by weight of magnesium stearate;
based on the total weight of granules and all extragranular ingredients together.

17. The pharmaceutical composition according to claim 1, comprising
(a) 15-70% by weight of granules which in turn comprise
10-40% by weight of the compound of formula (I);
60-90% by weight of copovidone; and
0.1-2% by weight colloidal silicon dioxide;
based on the total weight of the granules;
(b) 20-75% by weight of microcrystalline cellulose;
(c) 5-15% by weight of sodium carboxymethyl cellulose;
(d) 5-15% by weight of HPMC acetate succinate;
(e) 1-5% by weight of colloidal silicon dioxide; and
(f) 0.5-2% by weight of magnesium stearate;
based on the total weight of granules and all extragranular ingredients together.

18. The pharmaceutical composition according to claim 1, comprising
(a) 15-70% by weight of granules which in turn comprise
10-40% by weight of the compound of formula (I);
60-90% by weight of copovidone; and
0.1-2% by weight colloidal silicon dioxide;
based on the total weight of the granules;
(b) 20-75% by weight of microcrystalline cellulose;
(c) 5-18% by weight of sodium carboxymethyl cellulose;
(d) 5-15% by weight of HPMC acetate succinate;
(e) 1-5% by weight of colloidal silicon dioxide; and
(f) 0.5-2% by weight of magnesium stearate;
based on the total weight of granules and all extragranular ingredients together.

19. The pharmaceutical composition according to claim 1, comprising
(a) 15-70% by weight of granules which in turn comprise
10-40% by weight of the compound of formula (I);
60-90% by weight of copovidone; and
0.1-2% by weight colloidal silicon dioxide;
based on the total weight of the granules;
(b) 20-75% by weight of microcrystalline cellulose;
(c) 5-15% by weight of crospovidone;
(d) 1-5% by weight of colloidal silicon dioxide; and
(e) 0.5-2% by weight of sodium stearyl fumarate;
based on the total weight of granules and all extragranular ingredients together.

20. The pharmaceutical composition according to claim 1, wherein the composition is in the form of a capsule, tablet, or sachet.

21. A process for making the pharmaceutical composition as defined by claim 1, comprising a melt granulation or a melt extrusion step.

22. The process according to claim 21 further characterized by the following process steps:
(1) melt-granulating or melt-extruding, preferably melt-extruding the compound (4S,5R)-3-(2′-Amino-2-morpholin-4-yl-4′-trifluoromethyl-[4,5]bipyrimidinyl-6-yl)-4-hydroxymethyl-5-methyl-oxazolidin-2-one or any pharmaceutically acceptable salt thereof together with the polymer poly(N-vinylpyrrolidone) (PVP) or any derivative thereof, preferably, said polymer is a copolymer of N-vinylpyrrolidone and vinylacetate, and optionally colloidal silica;
(2) milling and/or screening the melt granulates obtained by step (1) to obtain melt granulates with a median particle size median particle size within 250 to 1000 μm, preferably within 300 to 750 μm, more preferably within 300 to 500 μm;
(3) blending the melt granulates obtained by step (2) with an anti-nucleating agent, preferably said anti-nucleating agent is an acrylic polymer or a cellulose derived polymer or combinations thereof, more preferably said anti-nucleating agent is selected from the group consisting of Eudragit L100, HPMC and HPMC-AS, even more preferably said anti-nucleating agent is hydroxypropyl methylcellulose acetate succinate (HPMC-AS), and optionally microcrystalline cellulose, and optionally a disintegrant, preferably, cross-linked poly(N-vinylpyrrolidone) or sodium carboxymethyl cellulose, and optionally a glidant, and optionally a lubricant;
(4) encapsulating or tabletting the blend obtained by step (3), preferably encapsulating said blend into hard gelatine capsules.

23. The process according to claim 21 further characterized by the following process steps:
(1) melt-granulating or melt-extruding, preferably melt-extruding the compound (4S,5R)-3-(2′-Amino-2-morpholin-4-yl-4′-trifluoromethyl-[4,5]bipyrimidinyl-6-yl)-4-hydroxymethyl-5-methyl-oxazolidin-2-one or any pharmaceutically acceptable salt thereof together with the polymer poly(N-vinylpyrrolidone) (PVP) or any derivative thereof, preferably, said polymer is a copolymer of N-vinylpyrrolidone and vinylacetate, and optionally colloidal silica;
(2) milling and/or screening the melt granulates obtained by step (1) to obtain melt granulates with a median particle size median particle size within 250 to 1000 μm, preferably within 300 to 750 μm;
(3) optionally blending the melt granulates obtained by step (2) with one or more excipients, including but not limited to microcrystalline cellulose, a disintegrant, preferably, crospovidone; a glidant, a lubricant, or a combination thereof; and
(4) encapsulating or tabletting the blend obtained by step (3), preferably tableting said blend into tablets.

24. A pharmaceutical composition obtainable by the process according to claim 21.

25. A pharmaceutical composition according to claim 1, for use in the treatment of cancer, for use in the treatment or suppression of tumors, or for use in the treatment or prevention of other conditions, diseases or disorders dependent on PI3K.
